(19) United States
(12) Patent Application Publication
(10) Pub. No.: US 2020/0375170 A1
(43) Pub. Date: Dec. 3, 2020

(54) VOLATILE MEDIUM DELIVERY DEVICE, SYSTEM, AND METHOD OF USE

(71) Applicant: TAKASAGO INTERNATIONAL CORPORATION, Tokyo (JP)

(72) Inventors: Thomas F. SANTINI, Doylestown, PA (US); Anthony R. BUDRAITIS, Hoboken, NJ (US)

(73) Assignee: TAKASAGO INTERNATIONAL CORPORATION, Tokyo (JP)

(21) Appl. No.: 16/080,168

(22) PCT Filed: Apr. 27, 2017
(86) PCT No.: PCT/US2017/029779

§ 371 Date: Aug. 27, 2018

Related U.S. Application Data
(60) Provisional application No. 62/328,328, filed on Apr. 27, 2016.

(51) Int. Cl. A01M 1/20; A61L 9/12; A61L 9/04

(52) U.S. Cl. CPC A01M 1/2055; A61L 2209/15; A61L 9/042; A61L 9/12

(57) ABSTRACT

A volatile medium delivery system, comprising a housing, a barrier element engageable with the housing, and a substrate having a volatile medium absorbed therein, wherein the substrate is engageable with the housing and positionable with respect to the housing to control a predetermined release rate of volatile medium to an external environment.

### CROSS-REFERENCE TO RELATED APPLICATION

This application claims priority to U.S. Provisional Application No. 62/328,328, filed on Apr. 27, 2016, which is incorporated herein by reference in its entirety.

### BACKGROUND OF THE DISCLOSED SUBJECT MATTER

### Field of the Disclosed Subject Matter

The disclosed subject matter relates to a volatile medium delivery device, system, and method of use.

### Description of the Related Art

Absorbent substrates that release volatile air treating components have found broad application across many industries due to the effective and economical nature of devices that utilize such substrates. Commonly, devices incorporating such substrates have been purposed for the diffusion of fragrance in a closed space in order to create an indoor fragrance experience. Additionally, these active fragrance compositions can be formulated with malodor reduction and odor neutralization agents for use in areas where malodors can accumulate. Such devices can also be used for the release of other volatile active compositions. For example, the diffusion of medicants, decongestants, insect repellants, insecticides, aroma therapeutic compositions, can use devices having an absorbent substrate as a releasing medium.

Paper can be used as an absorbent substrate for the release of these volatile active compositions. Paper can be a versatile substrate due to its variety of compositional and construction methods which in turn influence its ability to contain and release a volatile agent. This can offer a large degree of flexibility as to the type of device that incorporates such releasing platforms. Additionally, the economy of paper and its ability to be graphically enhanced to support a concept or a particular fragrance positioning makes it particularly well suited for these applications.

Devices that utilize an absorbent substrate can rely on a die cut form which is then dosed with the desired amount of fragrance. The substrate can then be sealed within a rigid plastic housing or contained within an impervious flexible packaging laminate which includes a barrier layer, such as polyester or metal foil. Because paper, as a releasing medium, exerts little influence on the rate of release of the volatile air treating composition absorbed within it, a method of adjustability can be provided to prevent the premature dissipation of the volatile compositions and extend the functional life of the unit. For example, as described in U.S. Pat. No. 4,014,501, an adjustable housing can be used that can open and close to vary the exposure to the absorbent substrate. Alternatively, a manipulable and flexible vapor impermeable overwrap can be provided that is engaged with the absorbent substrate such that it can be incrementally removed to expose a desired amount of the surface of the substrate. For example, as the exposed portion of the substrate depletes a fragrance, more surface can be exposed to provide a more uniform fragrance intensity experience for the user over the lifetime of the unit. However, this method can result in the barrier overwrap hanging from the unit and creating an unsightly appearance.

Both of the above methods of adjustability require the manipulation of either the adjustable housing or vapor impermeable overwrap in order to limit the exposure of the emanating substrate and to offer the user some measure of control over the amount of volatile composition that diffuses.

Accordingly, there remains a continued need for an efficient and economic system for delivering a volatile medium. The presently disclosed subject matter satisfies the above and other needs. Embodiments of the disclosed subject matter provide a device, system, and method of use that utilizes a device having an impregnated absorbent substrate that can release volatile air treating compositions, such as fragrances, and provides selectable control of releasing volatile compositions at predetermined release rates when using such device while being simple, efficient, and cost effective and while having an attractive appearance.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The presently disclosed subject matter provides novel techniques to increase the adjustability of an impregnated absorbent structure of a device. These techniques result in an economical, easy to manipulate, and attractive alternative to known delivery devices.

Accordingly, the presently disclosed subject matter provides methods to control the release of a volatile medium, e.g., an air treating composition such as fragrance, from an absorbent substrate. Such methods can be inexpensively implemented and provide adjustability without reliance on secondary packaging to offer opening and closing features to the delivery device.

The disclosed systems and methods can be used to deliver any volatile medium from an absorbent substrate. Such volatile mediums include, but are not limited to medicants, decongestants, insecticides, insect repellants, aroma therapeutic agents and the like. For these applications, the substrate can be easily and controllably oriented to regulate an amount of volatile medium released to an external environment based upon an amount of surface exposed for a defined period of time, amongst other factors such as a size dimension of the substrate, a material of the substrate, and a loading of the volatile medium therein.

Thus, the presently disclosed subject matter at least provides techniques for altering the exposure of an absorbent substrate to the surrounding atmosphere by manipulating the substrate into multiple orientations such that each orientation exposes a different predetermined amount of the surface of the substrate. These alternate configurations can permit an increase or decrease in the amount of exposure, thereby influencing a release rate of a volatile fluid medium to an external environment. Particularly, the amount of the surface of the substrate that is exposed can be a determinant factor in the release rate for a given volatile fluid medium, once environmental conditions such as temperature are excluded.

Accordingly, the presently disclosed systems and methods can offer an option of selecting an orientation of the substrate that influences an amount of surface area of the substrate which is exposed. Consequently, the release rate of the volatile fluid medium can be controlled by adjusting the available surface area of the releasing substrate within the unit to meet a desired preference for intensity, thereby resulting in a more satisfying user experience.

To achieve these and other advantages and in accordance with the purpose of the disclosed subject matter, as embodied and broadly described, the disclosed subject matter includes a volatile medium delivery system. The volatile medium delivery system includes a housing, a barrier element engageable with the housing, and a substrate having a volatile medium absorbed therein, where the substrate is engageable with the housing and positionable with respect to the housing to control a predetermined release rate of volatile medium to an external environment.

As embodied herein, the housing can further include a bottom surface, a sidewall coupled to the bottom surface, and a support device, where the substrate is engageable with the support device. In certain embodiments, the sidewall further includes a flange and the barrier element is engageable with the housing about the flange. For example, the barrier element can be removably coupled to the flange by heat seal.

In some embodiments, at least a portion of the substrate is engageable with the sidewall. The sidewall can further include a projection and the substrate can be engageable with the projection.

As embodied herein, the support device can include at least a first structure and a second structure, where the first structure is disposed a predetermined distance dimension from the second structure to define a first channel therebetween, and where the substrate is receivable in the channel. The predetermined distance dimension can include any suitable dimension to accommodate at least a width or thickness of the substrate. For embodiments where two members of the substrate are positioned within the support device, the predetermined distance dimension of the first channel is at least twice the width of the substrate, such as when the substrate is folded upon itself and the members of the substrate are each disposed within the support device, as discussed herein. As such, a thickness dimension of the substrate is not greater than the predetermined distance dimension of the channel of the support device that supports the substrate. In certain embodiments, the first structure includes a first wall and a second wall disposed the predetermined distance dimension from the first wall, where the second structure includes a first wall and a second wall disposed from the first wall of the second structure at the predetermined distance dimension, where each of the first wall of the first structure, the second wall of the first structure, the first wall of the second structure, and the second wall of the second structure have an L-shaped configuration to define a channel in the shape of a plus (+) symbol.

As embodied herein, the substrate can include a first member and a second member, where each of the first member and second member has a front surface and a back surface. In certain embodiments, the first member can be coupled to the second member by a hinge. In certain embodiments, the first member defines a slit, where the first member is engageable with the second member by insertion of the second member into the slit of the first member.

For example, the substrate can be positioned in the support structure having the front surface of the first member and the front surface of the second member exposed to the external environment to control the predetermined release rate of volatile medium. In certain embodiments, the substrate is positioned in the support device having the front and back surfaces of the first member and the front and back surfaces of the second member exposed to the external environment to effect a maximum release rate of volatile medium.

As embodied herein, the housing can further include a handle device, where the handle device defines an aperture. Alternatively or additionally, the volatile medium delivery system can include a frame member, where the substrate is receivable in the frame member. The frame member can include a hinge.

The volatile medium can be at least one of a fragrance, insecticide, medicant, decongestant, insect repellant, or a combination thereof. The substrate can include at least one of paper, wood, polyethylene, fabric, foam, paper substitute, absorbent ceramic, or a nonwoven material. In certain embodiments, the volatile medium can be emanated from the substrate to the external environment at the predetermined release rate for any variable length of life as desired by the consumer. Examples of suitable release rates can include at least one of 1 hour, 24 hours, 7 days, 14 days, 21 days, or 28 days dependent upon factors such as a size dimension of the substrate, a material of the substrate, and a loading of the volatile medium therein.

In accordance with another aspect of the disclosed subject matter, a method of using a volatile medium delivery system is provided. The method includes providing a volatile medium delivery device including a housing and a substrate having a volatile medium absorbed therein. The method further includes engaging the substrate with the housing and positioning the substrate with respect to the housing to control a predetermined release rate of volatile medium to the external environment.

In certain embodiments, the substrate includes a first member and a second member, where each of the first member and second member have a front surface and a back surface, and where the positioning includes engaging at least a portion of the substrate with the housing to expose at least one of the first member and the second member to the external environment.

It is to be understood that both the foregoing general description and the following detailed description and drawings are examples and are provided for purpose of illustration and not intended to limit the scope of the disclosed subject matter in any manner.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

The subject matter of the application will be more readily understood from the following detailed description when read in conjunction with the accompanying drawings, in which:

FIG. 1 provides a perspective view of a volatile medium delivery system in accordance with an embodiment of the presently disclosed subject matter.

FIG. 2 provides a perspective view of the volatile medium delivery system of FIG. 1 without an attached barrier element.

FIG. 2B provides a perspective view of the volatile medium delivery system with the substrate in a partially opened configuration.

FIG. 3 depicts a substrate in accordance with the presently disclosed subject matter.

FIG. 4 provides a perspective view of the substrate of FIG. 3 engaged with a housing in accordance with the presently disclosed subject matter.

FIG. 5 provides a perspective view of a housing including a support device in accordance with the presently disclosed subject matter.

FIGS. 5B-5E depict a cross-sectional view of a housing having various support device configurations in accordance with embodiments of the presently disclosed subject matter.

FIG. 6 provides a perspective view of a substrate coupled with the support device of FIG. 5, in accordance with the presently disclosed subject matter.

FIG. 7 provides a perspective view of a substrate coupled with the support device of FIG. 5 in another configuration in accordance with the presently disclosed subject matter.

FIG. 8 depicts a substrate in accordance with an embodiment of the presently disclosed subject matter.

FIG. 9 provides a perspective view of the substrate of FIG. 8 engaged with a housing in accordance with the presently disclosed subject matter.

FIG. 10 provides a perspective view of a substrate coupled with the support device of FIG. 9 in a configuration in accordance with the presently disclosed subject matter.

FIG. 11 provides a perspective view of a substrate coupled with the support device of FIG. 9 in another configuration in accordance with the presently disclosed subject matter.

FIG. 12 provides a perspective view of another housing including a support device in accordance with the presently disclosed subject matter.

FIG. 13 depicts another substrate in accordance with an embodiment of the presently disclosed subject matter.

FIG. 14 provides a perspective view of a substrate of FIG. 13 coupled with the support device of FIG. 12 in another configuration in accordance with the presently disclosed subject matter.

FIG. 15 provides a perspective view of a volatile medium delivery system having a frame member in accordance with an embodiment of the presently disclosed subject matter.

FIG. 16 provides a perspective view of the volatile medium delivery system of FIG. 15 having the substrate in an open configuration in accordance with the presently disclosed subject matter.

FIG. 17A provides a front view of a volatile medium delivery system having a handle device in accordance with an embodiment of the presently disclosed subject matter.

FIG. 17B provides a front view of a volatile medium delivery system having a housing with a projection in accordance with an embodiment of the presently disclosed subject matter.

FIG. 18 provides a perspective view of FIG. 17B such that the substrate is partially unfolded in accordance with the presently disclosed subject matter.

FIG. 19 depicts a perspective view of the delivery device system including a lid in accordance with the presently disclosed subject matter.

FIG. 20 depicts the system of FIG. 19 with the lid engaged with the housing.

FIG. 21 depicts a perspective view of the delivery device system of FIG. 20 including a transparent lid in accordance with the presently disclosed subject matter.

FIG. 22 depicts a perspective view of a substrate having a flower shape within the housing in accordance with the presently disclosed subject matter.

FIG. 23 depicts a perspective view of a substrate having a graphic within the housing in accordance with the presently disclosed subject matter.

### DETAILED DESCRIPTION

Reference will now be made in detail to embodiments of the disclosed subject matter, an example of which is illustrated in the accompanying drawings. The disclosed subject matter will be described in conjunction with the detailed description of the system.

The presently disclosed subject matter relates to volatile medium delivery devices which utilize an absorbent substrate functioning as an emanating element that can be manipulated into a variety of configurations to control the evaporative release of the absorbed volatile medium. The ability to orient the substrate into alternate configurations to vary the degree of exposure of the releasing surface of the substrate can be facilitated by the housing which is configured to engage the substrate such that multiple orientations are possible.

In accordance with the disclosed subject matter, the disclosed subject matter provides a volatile medium delivery system. The volatile medium delivery system includes a housing, a barrier element engageable with the housing, and a substrate having a volatile medium absorbed therein, where the substrate is engageable with the housing and positionable with respect to the housing to control a predetermined release rate of volatile medium to an external environment.

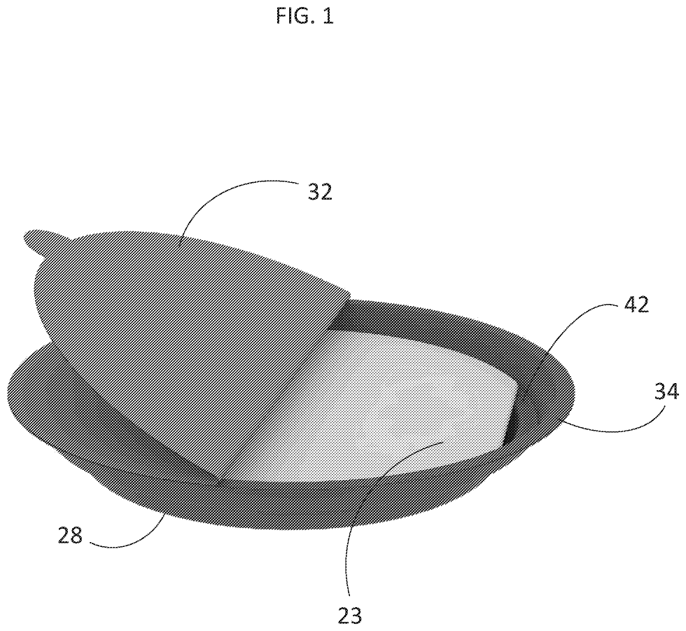

Solely for purpose of illustration, an embodiment of a volatile medium delivery system is shown schematically in FIG. 1. The examples herein are not intended to limit the scope of the disclosed subject matter in any manner. Particularly, and as illustrated, the volatile medium delivery system includes a housing 28, a barrier element 32, and a substrate 23.

As embodied herein, the housing 28 can include a bottom surface and a sidewall coupled to the bottom surface. The sidewall can be coupled to the perimeter of the bottom surface and define an outer boundary of the housing 28. For example, and not limitation, the bottom surface and the sidewall can be integrally formed from the same material. As embodied herein, the housing 28 can be formed from a vapor impermeable material, and as such, can be used to contain a volatile medium therein. The housing can be manufactured in any suitable manner such as but not limited to thermoforming, injection molding, and metal stamping.

As embodied herein, the barrier element 32 is engageable with the housing 28. The barrier element and the housing can be engageable in a plurality of suitable manners. For example and with reference to FIG. 1, the sidewall of the housing 28 can further include a flange 34 that is disposed along an upper rim of the housing 28 and can extend at an angle from the housing 28. The angle can be any suitable dimension such as but not limited to approximately 45° to approximately 135° with respect to the sidewall. As such, the barrier element 32 can be engageable with the housing about the flange 34.

The barrier element 32 can be formed from any suitable vapor impermeable material and can engage with the housing 28 to form a vapor-tight seal. In this manner, the housing 28 together with the barrier element 32 can create an imperviously sealed unit that contains volatile medium. The substrate 23 can be packaged within the housing 28 with the volatile medium disposed therein. In this manner, the substrate 23 can be dosed with the volatile medium after the sealing of the barrier element by movement of the device, as further discussed herein. Alternatively, the substrate can be predosed with volatile medium and then disposed in the housing. By way of example, and not limitation, the barrier element 32 can be coupled to housing 28 by any known methods, such as but not limited to heat sealing. Where the housing 28 includes a flange 34, the barrier element 32 can be coupled to the flange 34 by heat seal. The heat seal can provide a detachable seal, such that the barrier element 32 is removably coupled to the housing 28. For example, as depicted in FIG. 1, the barrier element 32 can be peeled back or otherwise detachable from the housing 28. Additionally or alternatively to the barrier element 32, a removable, impervious lid can be coupled with the housing by any suitable construction, such as an interference fit as further discussed herein.

By way of example, and not limitation, the barrier element 32 can be formed of a flexible material. As such, the barrier element 32 can be partially removed from the housing 28 to expose a portion of the substrate 23, as depicted in FIG. 1. For example, the barrier element 32 can be made of a foil or a laminate material, or a polyester laminate material such as mylar or polyethylene terephthalate (PET).

As described above, the volatile medium delivery system can further include a substrate 23. As embodied herein, the substrate 23 can absorb a volatile medium that is present within the housing 28, or can be predosed with volatile medium. As will be described in greater detail below, the substrate 23 can be formed as a unitary piece which can optionally be folded onto itself. Alternatively or additionally, the substrate can include a plurality of elements that can be stacked within the housing 28. Additionally, the substrate 23 can have any suitable shape or size that can be accommodated within the housing 28.

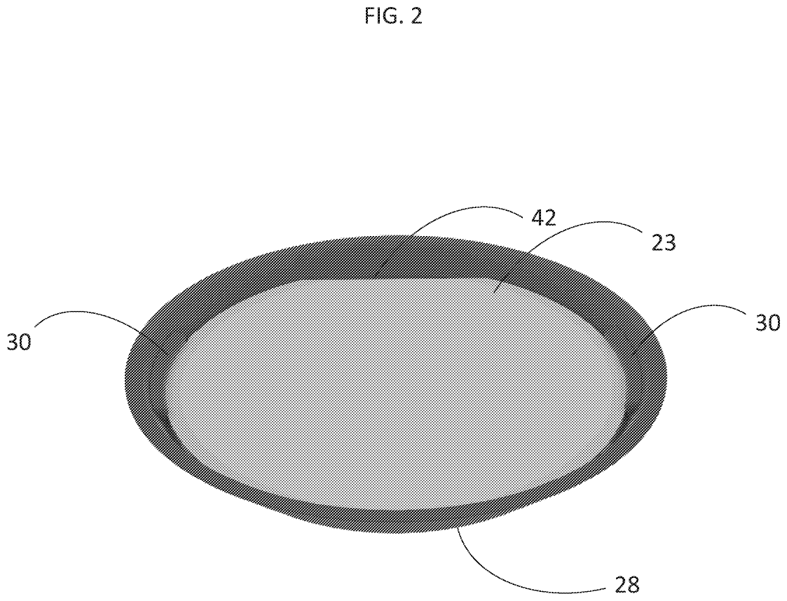

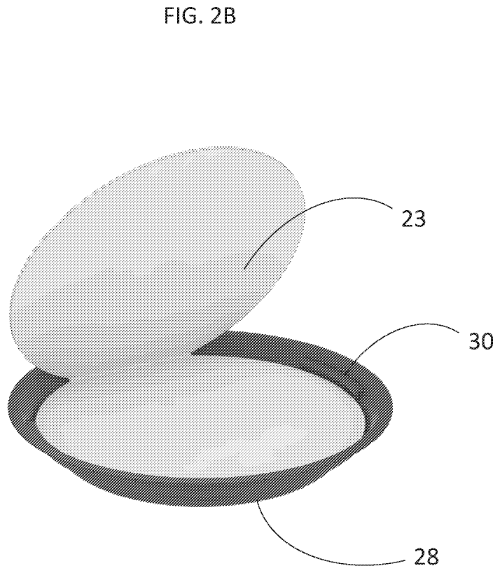

As embodied herein, the substrate 23 can be engageable with the housing 28 such as engageable with the sidewall of the housing. With continued reference to FIG. 1, the sidewall can additionally include a shelf 42. For example, the shelf 42 can be formed within the sidewall and disposed about the housing circumferentially. As such, the shelf 42 can extend along the entire perimeter of the sidewall, or along at least a portion of the perimeter. As embodied herein and as shown in FIG. 2, the substrate 23 can rest along the shelf 42 when packaged within the housing 28. In this manner, there can be an elevation of the substrate 23 in the housing from the bottom of the housing 28 when in the delivery configuration.

For purpose of illustration, another embodiment of a volatile medium delivery system having the barrier element removed is shown schematically in FIG. 2. With reference to FIG. 2, the sidewall of the housing 28 can include additional features to engage the substrate 23. For example, the sidewall can include one or more projections 30. The projection(s) 30 can alternatively include any outwardly extending device, such as a nub, rib, protuberance, and the like. Such devices can provide an interference fit with the substrate 23 to remain temporarily locked in place in the delivery configuration. In FIG. 2, the substrate 23 is shown folded onto itself and compressibly engaged into the housing 28 and disposed beneath projection 30. For example, the housing 28 can include both a shelf, as described in connection with FIG. 1, and one or more projections 30, such that the substrate can be packaged between the shelf 42 and the projection(s) 30. This delivery configuration can secure the substrate 23 within the housing 28 until the substrate 23 is removed from the housing 28. In this manner, the housing 28 can serve as the base that seals the volatile medium and the substrate therein, and the barrier element can function as the top to seal the volatile medium and substrate therein. Alternatively or additionally, at least one member of the substrate can remain in the delivery configuration and engaged with at least one projection 30, while permitting a second member of the substrate to pivoting away from the housing 28 as shown in FIG. 2B.

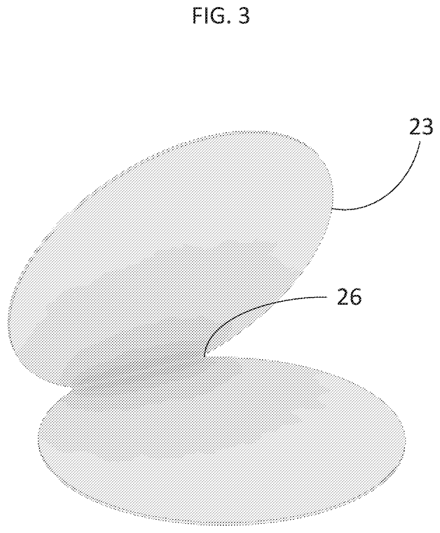

FIG. 3 provides a substrate for use in the presently disclosed subject matter. As is illustrated in FIG. 3, the substrate 23 can be formed as a monolithic structure. For example, the substrate 23 can be die cut from a larger sheet. In some embodiments, the substrate 23 can be folded onto itself such that the substrate 23 includes two members joined by a hinge 26, as shown in FIG. 3. For example, the substrate 23 can be folded at a midpoint to allow each member of the substrate 23 to have the same profile such that, when folded, the members align to create a continuous circumference. As such, the hinged substrate, as depicted in FIG. 3, can be positioned in a folded or wholly or partially unfolded manner to vary the amount of exposed surface. The substrate can additionally include a relief line to promote additional bending about the relief line to allow for further orientations of the substrate, as further discussed herein. The substrate 23 can be additionally engageable with the housing 28 upon being removed from the delivery configuration within the housing 28. As will be described in further detail below, features of the housing can facilitate the position of the substrate as shown in the figures.

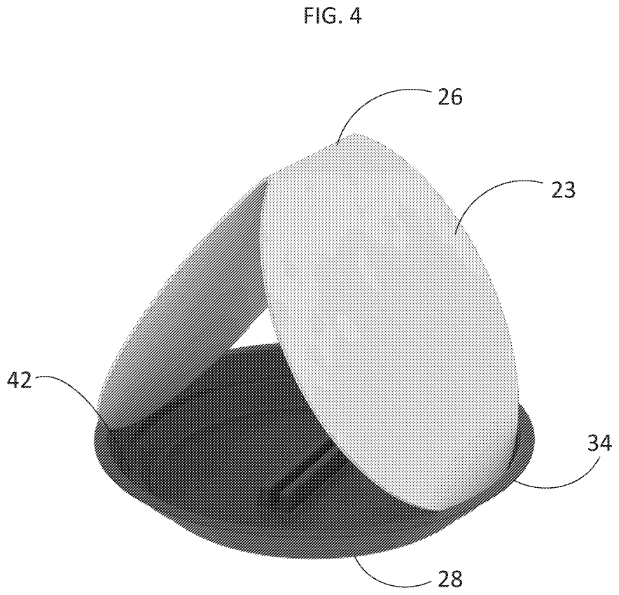

For illustration, FIG. 4 provides a configuration of a volatile medium delivery system where the substrate 23 is positioned in an inverted V configuration (\hat{ }) within the housing. In FIG. 4, the substrate 23 is removed from the delivery condition within the housing 28 and positioned in the inverted V configuration (\hat{ }) within the housing 28. Edges of the substrate 23 engage the housing 28 to stabilize the orientation. Where the housing 28 includes a shelf 42, as depicted in FIG. 4, the substrate 23 can engage the housing about the shelf 42. When the housing 28 includes a projection (not pictured in FIG. 4), the substrate 23 can engage the projection. As depicted in FIG. 4, all four surfaces of the substrate are exposed by inverting the orientation and by having the hinge 26 disposed away from the housing such that the bottom edges of the substrate 23 engage with the sidewalls of the housing 28. In this manner, the substrate 23 can be positioned in such partially unfolded manner to control the release rate of a volatile medium.

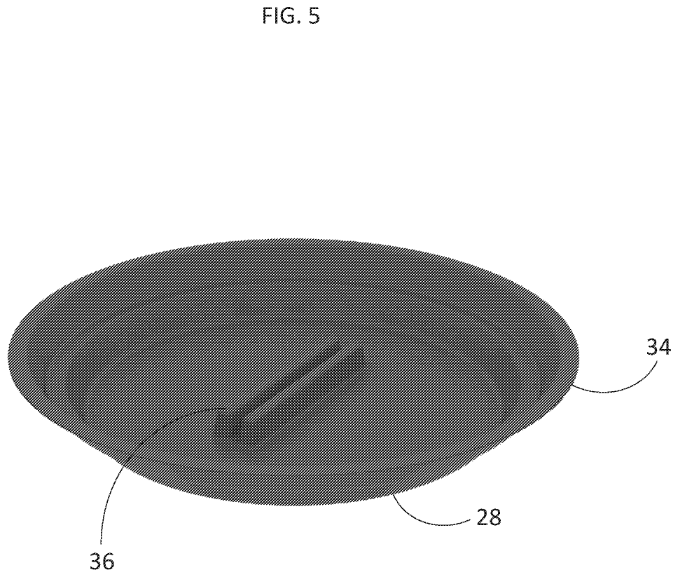

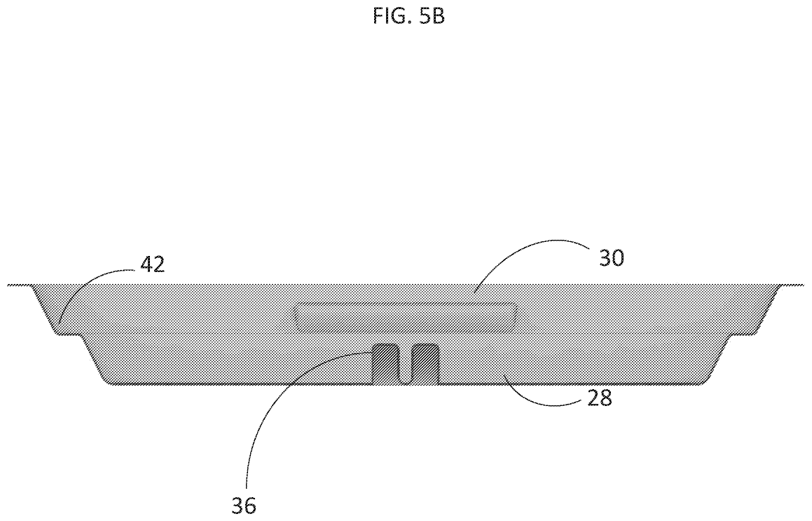

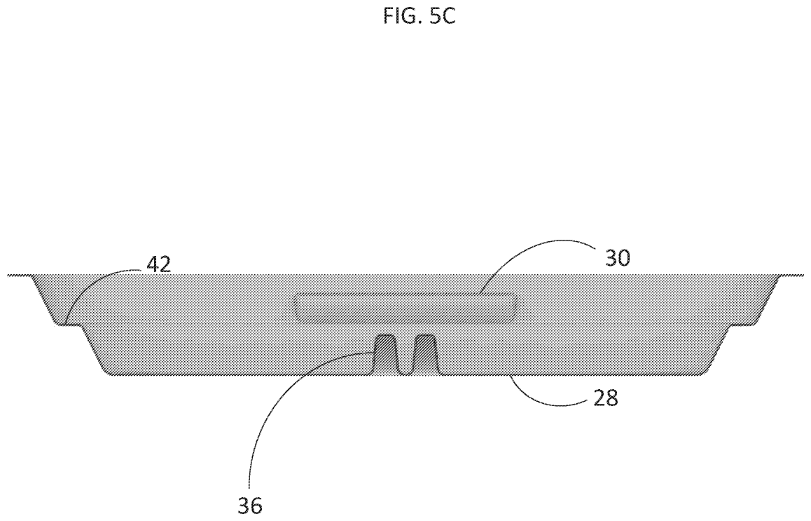

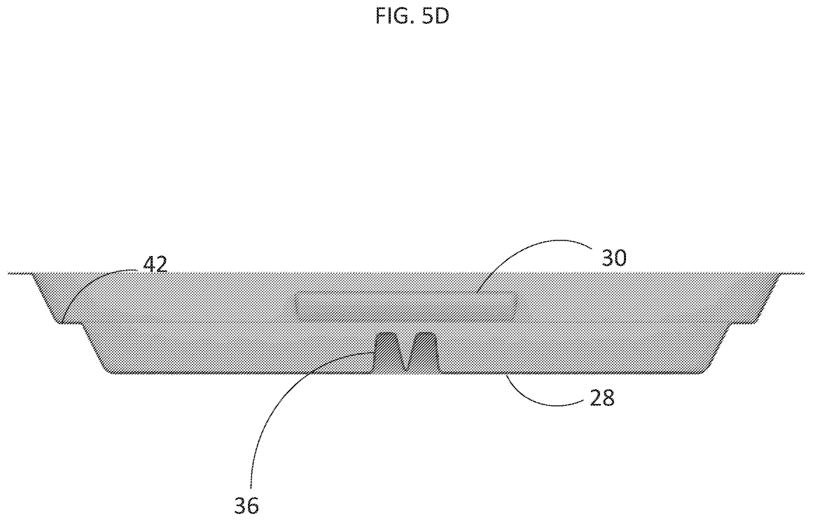

As embodied herein, the housing can include additional features that can be engaged by the substrate to position the substrate in other configurations. For illustration, FIG. 5 provides a housing 28 having a support device 36. As depicted in FIG. 5, the support device 36 can include two structures such as rails, which can be disposed adjacent each other. The first rail can be disposed a predetermined distance dimension from the second rail to define a channel between the two rails. FIG. 5B-FIG. 5D depict cross-sectional views of different embodiments of the housing 28 having support devices of various suitable profiles. As shown in FIG. 5B, the longitudinal sides of the rails can be approximately perpendicular to the bottom surface of the housing and the rails can have approximately rectangular profiles that define a channel therebetween. Alternatively in FIG. 5C, the rails can be chamfered with the bottom surface. FIG. 5D depicts a cross-sectional profile of a support device having a trapezoidal profile defining a channel having a V configuration. The length dimension between the first and second rail structures can at least correspond to a thickness of the substrate, such that the substrate is receivable within the channel. In embodiments having a channel with a V configuration the widest part of the channel must at least accommodate a thickness of the substrate, For example, certain paper substrates can have a thickness from approximately 0.020 inches to approximately 0.065 inches. A corresponding channel to support such paper substrate can have a thickness from approximately 0.020 inches to approximately 0.130 inches, in order to accommodate one or both members of the substrate. FIG. 5E depicts an alternative support device without rail structures. In this embodiment, the bottom surface of the housing defines a recessed channel 36′ therein to receive and support a substrate selectively disposed within the recessed channel.

As embodied herein, the support device 36 can be formed in the base of the housing 28 and can support the substrate therein. Upon being removed from the housing 28 in its delivery configuration, the substrate can be engaged with the support device 36 and can be positioned in various orientations as described herein. Although not illustrated in FIG. 5, the support device 36 may be used as a rest for the substrate 23 and support the substrate from beneath when the substrate is packaged within the housing 28 in the delivery configuration, if desired.

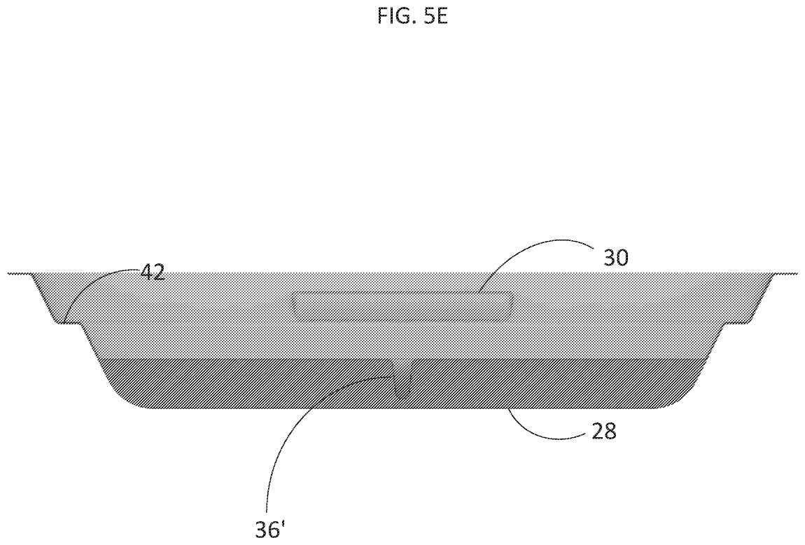

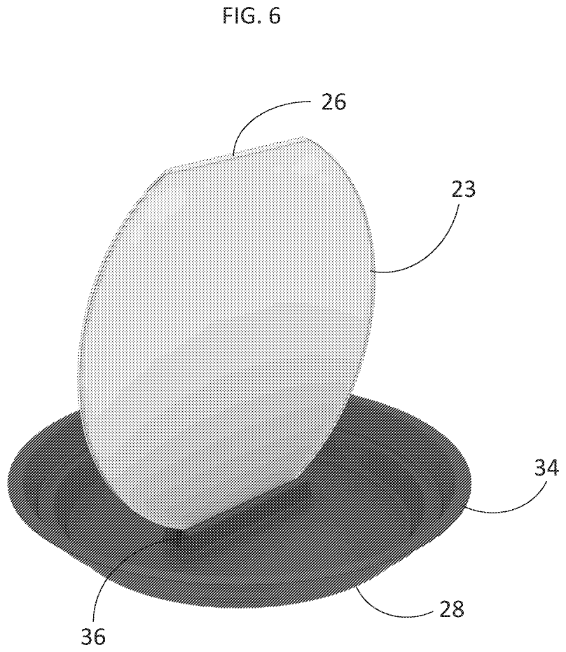

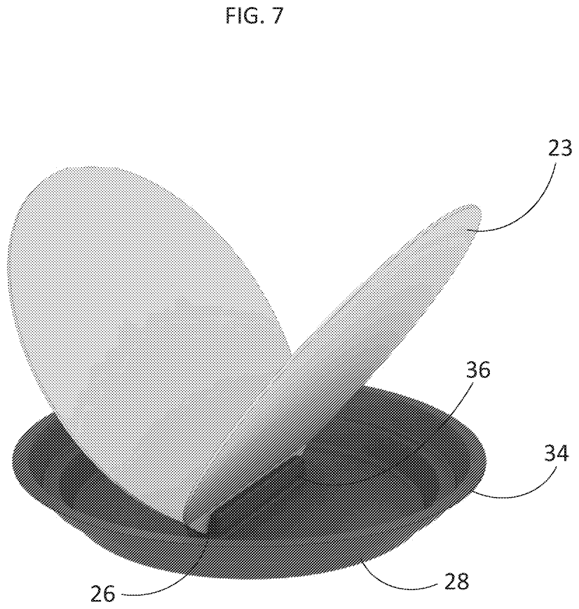

According to the disclosed subject matter, the substrate can be positioned in the support device 36 in various configurations to control the release rate of the volatile medium. For purpose of illustration, FIGS. 6 and 7 provide additional configurations of the substrate of FIG. 3 engaged with the housing FIG. 5. With reference to FIG. 6, the substrate 23 can be folded onto itself along a hinge 26 and inserted in the support device 36 such that the hinge 26 is disposed away from the housing. In this manner, the hinge 26 can secure the substrate 23 in a folded position and the support device 36 can keep the substrate in a closed position. In this configuration, only two surfaces of the substrate 23, i.e., one surface of each member of the substrate is exposed to the environment, which controls the predetermined release of volatile medium to the external environment. The unexposed surfaces can be later exposed upon changing the configuration of the substrate.

Alternatively, with reference to FIG. 7, the substrate 23 can be inserted into the support device 36. As such, the two members of the substrate 23 can be positioned spaced apart into a partially unfolded position. In this manner, all four surfaces of the substrate can be fully exposed for a greater release rate than that of FIG. 6. In some embodiments as previously noted, one or both members of the substrate 23 can include a line of relief adjacent to the hinge 26. In this manner, while the hinge 26 is anchored in the support device 36, the members can be additionally folded along the lines of relief to ensure that the substrate 23 remains in the open configuration, i.e., that all four surfaces remain exposed.

Accordingly, the amount of adjustability provided by the presently disclosed subject matter can be evident by the progression of exposed area of the substrate in the various configurations. As more of the substrate is exposed, more surface area of the substrate is exposed and greater volatile medium can be available for release. For example, by choosing among configurations having one exposed surface (see FIG. 2), two exposed surfaces (see FIG. 6), and four exposed surfaces (see FIGS. 4 and 7), the release rate of a volatile medium can be controlled with increasing release rates, as desired.

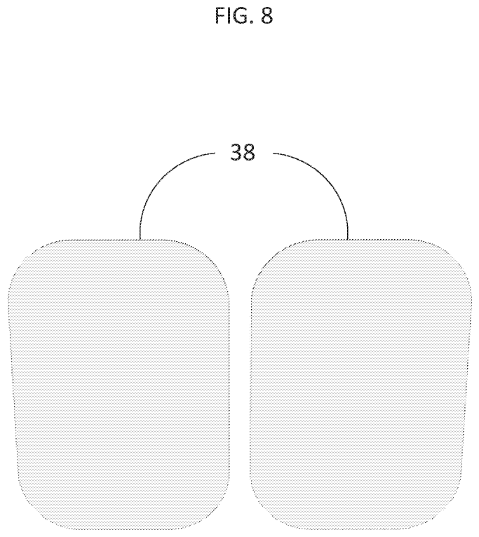

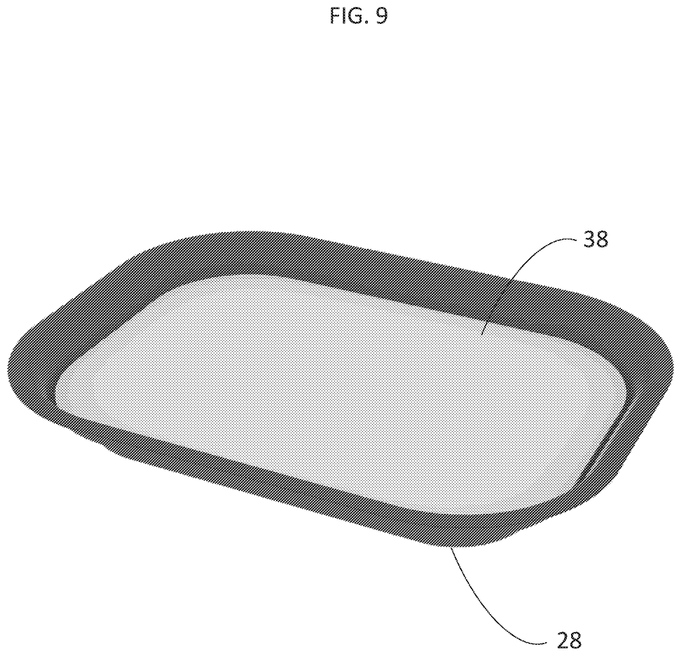

As embodied herein, it is also possible to achieve the same benefit of release rate of multiple positions, as illustrated in the foregoing description, by using a substrate having two separately formed members instead of the monolithically formed substrate as depicted in FIG. 3. For example, FIG. 8 provides an illustration of a substrate 38 having two discrete members, which can be die cut from a larger substrate. FIGS. 9 through 11 illustrate configurations of the substrate 38 within a housing, similar to the descriptions above with respect to FIGS. 5-7.

With reference to FIG. 9, two discrete members of substrate 38 are stacked, one on top of the other, within a housing 28. Accordingly, the substrate 38 can be packaged in the housing 28, as described previously. As such, the housing 28 can have any and all of the features described above including at least a projection and a shelf. For example, the housing 28 can be coupled to a barrier element (not pictured in FIG. 9) to provide an impervious seal therebetween to contain a volatile medium and substrate therein. The barrier element can be removed or partially pulled back to expose one surface of the substrate 38, as depicted in FIG. 9.

As embodied herein, the housing 28 of FIG. 9 can include a support device 36, as described above. Thus, the individual members of the substrate 38 can be disposed within the support device 36 to achieve the positions described above in connection with the singularly formed substrate with respect to FIGS. 3 through 7.

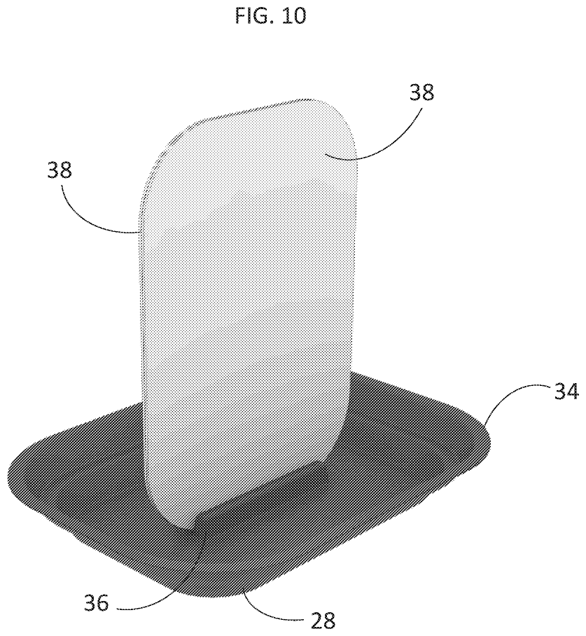

For the purpose of illustration, FIG. 10 depicts the members of the substrate 38 in an upright position such that the substrate is approximately perpendicular to the bottom surface of the housing. Both members of the substrate have been inserted into the support device to provide two sides of exposure. Although in FIG. 10, the members are depicted in the same orientation, one member could be rotated a predetermined degree of orientation to increase the amount of surface of the substrate 38 that is exposed.

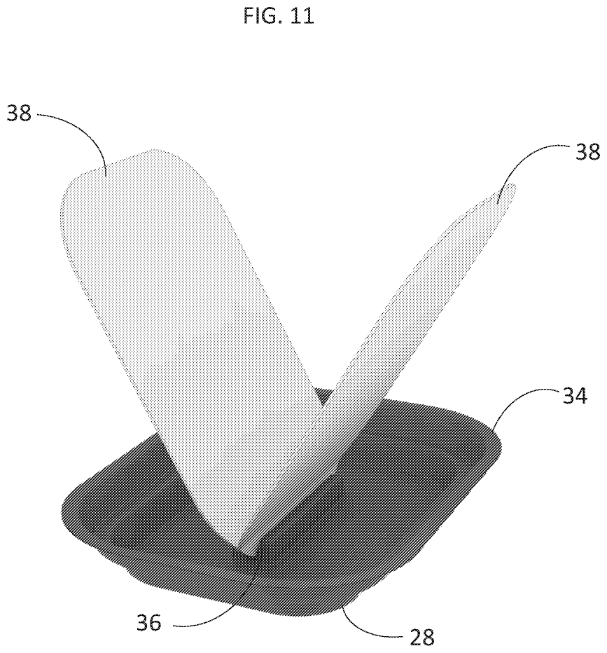

Alternatively, with reference to FIG. 11, the members of the substrate 38 can be disposed open to expose all four surfaces of the substrate. The bottoms of each member can be anchored within the support device 36. In certain embodiments, each member can include a line of relief adjacent to the bottom edge. When the bottoms of the members are anchored within the support device 36, the members can be folded along the line of relief to ensure that the substrate 38 remains in the open configuration.

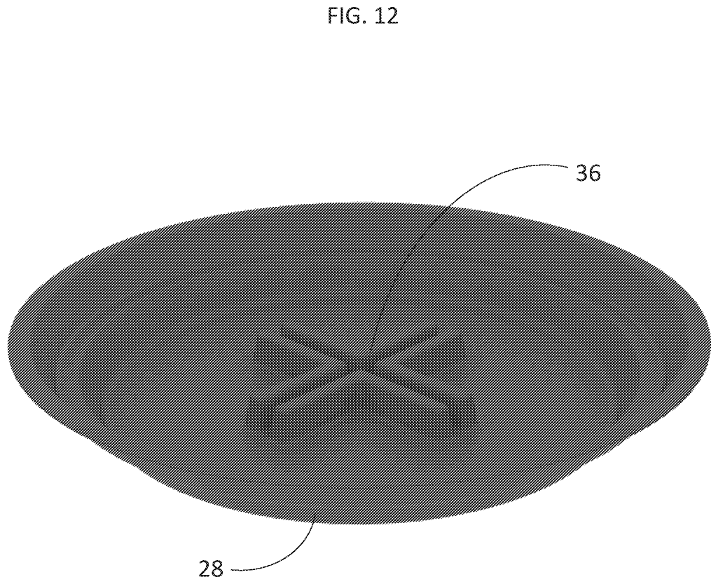

As embodied herein, the support device can define a channel in the shape of a plus (+) symbol. For purpose of illustration, FIG. 12 provides a housing having such at least one channel. For example, the first structure of the support device 36 can include two L-shaped walls and the second structure of the support device 36 can likewise include two L-shaped walls. Each pair of L-shaped walls can be separated by a predetermined distance dimension, as described above, and collectively positioned to form a shape of a plus-symbol to define at least one channel as shown.

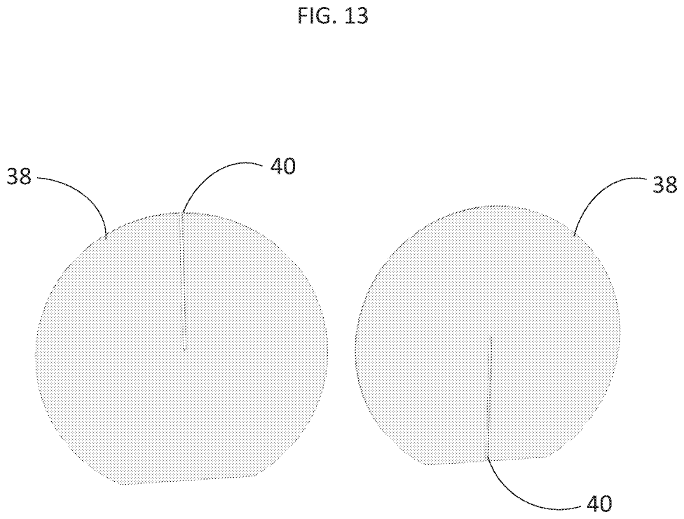

For the purpose of illustration, FIG. 13 depicts a substrate that can engage with the housing of FIG. 12. As shown in FIG. 13, each member of a substrate 38 can define a slit 40. Accordingly, one member can be slid down the center of the other member such that the members lockably engage to form a three-dimensional globe orientation. Although the embodiment of FIG. 13 shows a substrate where both members define a slit, a similar result can be achieved where only one member defines a slit.

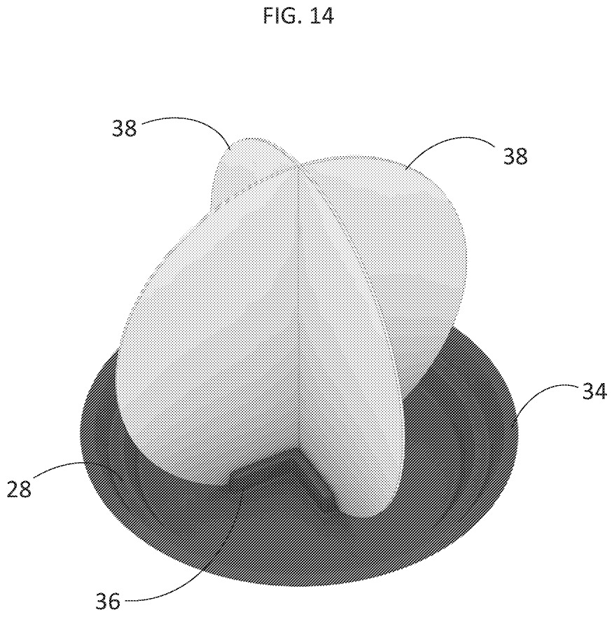

For illustration, FIG. 14 depicts the three-dimensional upright globe orientation of the substrate of FIG. 13. As shown in FIG. 14, all four surfaces of the substrate can be exposed by locking the two members together and inserting the members into a support device 36 that is configured to receive all four bottom edges of the substrate 38. In this manner, all four surfaces of the substrate 38 can be exposed, allowing a maximum release rate of a volatile medium from the substrate.

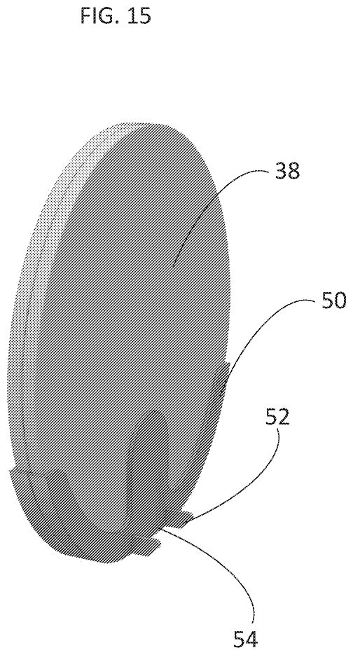

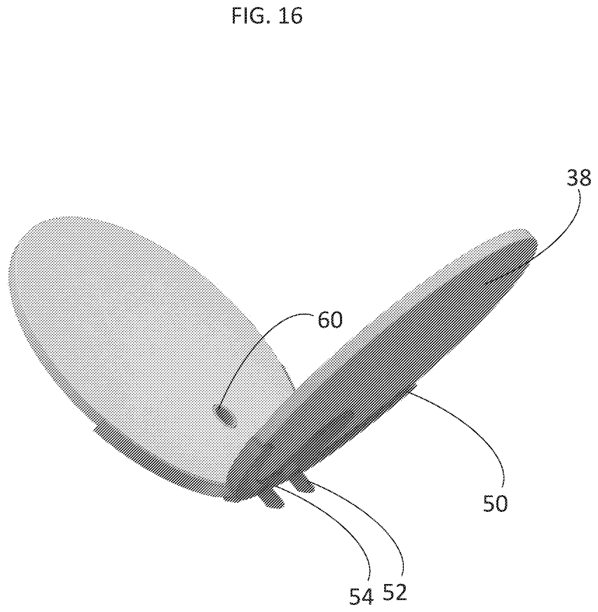

As embodied herein, the presently disclosed subject matter further provides a volatile medium delivery system including a frame member, where the substrate is receivable in the frame member. The frame member and substrate can be engageable with a housing as previously described above. For the purpose of illustration, FIGS. 15 and 16 provide a volatile medium delivery system including a frame member 50. As depicted in FIG. 15, the frame member 50 can define a recess, and the substrate 38 can be slidably received within the frame member in the recess. The substrate 38 can be monolithically singularly formed or include multiple separately formed members, as described above. In the embodiment of FIG. 15-16, the substrate comprises an absorbent ceramic material and the substrate includes a desired predetermined thickness associated with a desired void volume to capture volatile medium therein. In certain embodiments, the frame member 50 can include two or more feet 52 to support the frame member in an upright orientation. The feet 52 can be insertable within a support device of a housing or can straddle the support device (not shown).

With reference to FIG. 16, the frame member 50 can include a hinge 54. The substrate 38 can be rotated around the hinge to expose either two surfaces (see FIG. 15) or four surfaces (see FIG. 16). In certain embodiments, the frame member 50 can include male and female mating elements that can extend through the substrate 38. For example, as shown in FIG. 16, one side of the frame member can include a female mating element 60 that can define a recess in one member of the substrate 38. The other side of the frame member can include a corresponding male mating element (not pictured) that can protrude through the second member of the substrate 38. The male and female mating elements can engage when the frame member 50 is in a closed position (see FIG. 15) to ensure that only two surfaces of the substrate 38 are exposed. As such, in the embodiment of FIG. 16, the substrate defines a hole that corresponds to the mating elements.

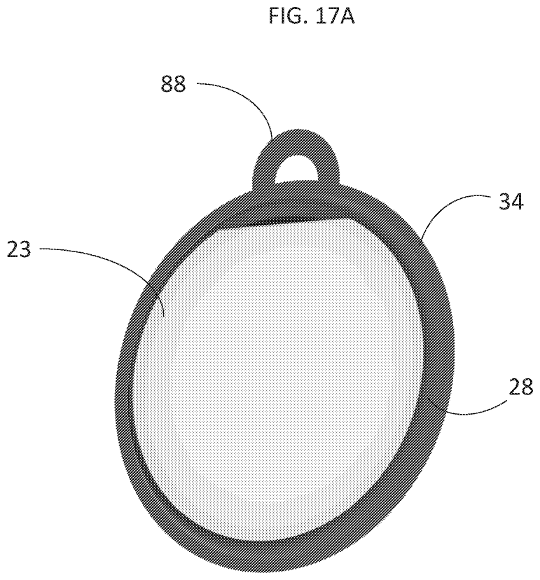

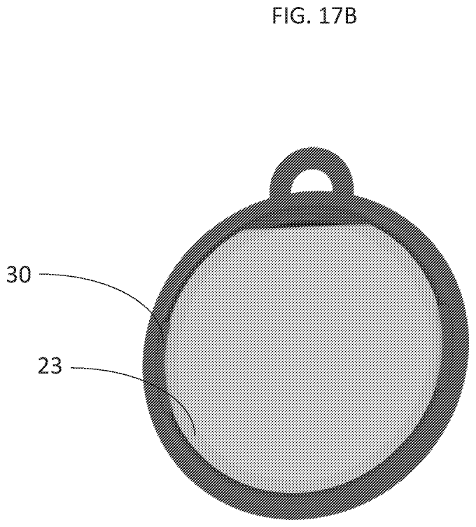

As embodied herein, the volatile medium delivery system of the presently disclosed subject matter can be placed on a horizontal surface after removing the barrier element to expose the substrate. Alternatively, the volatile medium delivery system can be positioned upright and suspended. For purpose of illustration, FIG. 17A provides a volatile medium delivery device that can be positioned upright and suspended. For example, as shown in FIG. 17A, the housing 28 can include a handle device 88 defining an aperture. The handle device 88 can be disposed in a non-sealing area of the flange 34 such that the handle device 88 does not interfere with the seal between the housing 28 and the barrier element (not pictured). Using the aperture, the unit can be suspended, e.g., hung from a hook, either directly or by looping a string or the like through the aperture. Additionally, in certain embodiments, the housing 28 can further include one or more projections, as depicted in FIG. 17B. The projections can be used to contain the substrate 23 within the housing 28 when the system is positioned upright.

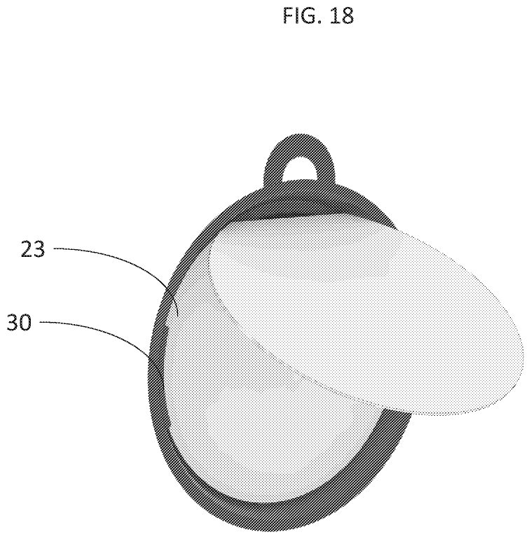

In embodiments where the volatile medium delivery system is positioned upright, it is possible to control the release rate of the volatile medium without removing the substrate and positioning it within the housing. For example, the barrier element can be partially removed to expose only a portion of the surface of the substrate. Alternatively or additionally, the housing can be coupled to a barrier element on either side of the housing such that a bottom surface of the housing is replaced by an additional barrier element. In this manner the release rate of the volatile medium can be controlled by choosing whether to expose both sides or a single side of the volatile medium. As shown in FIG. 18, at least one member of the substrate can remain in the delivery configuration, while permitting a second member to pivoting away from the housing.

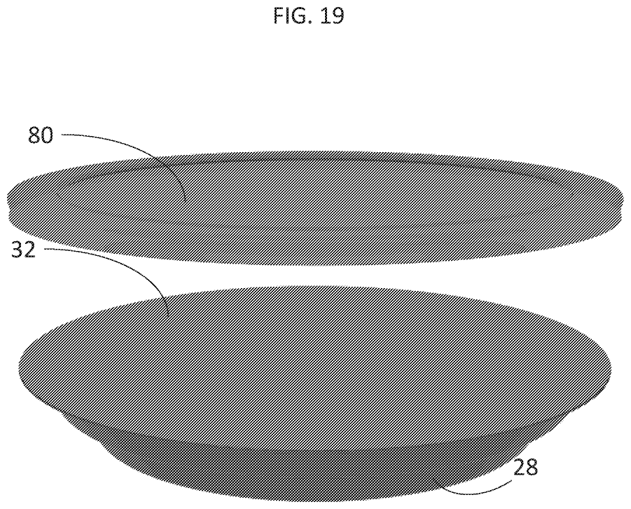

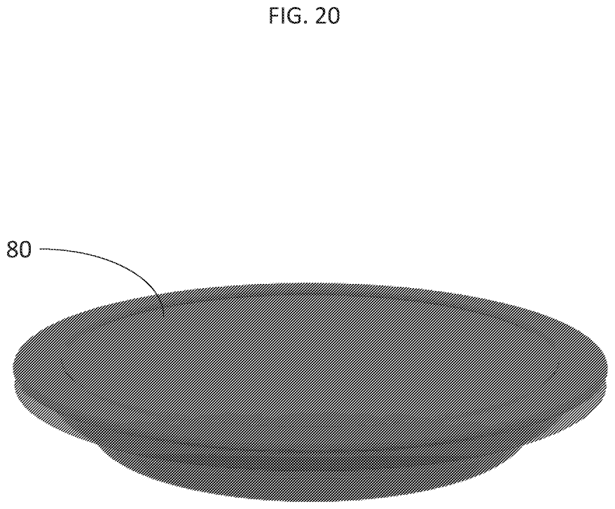

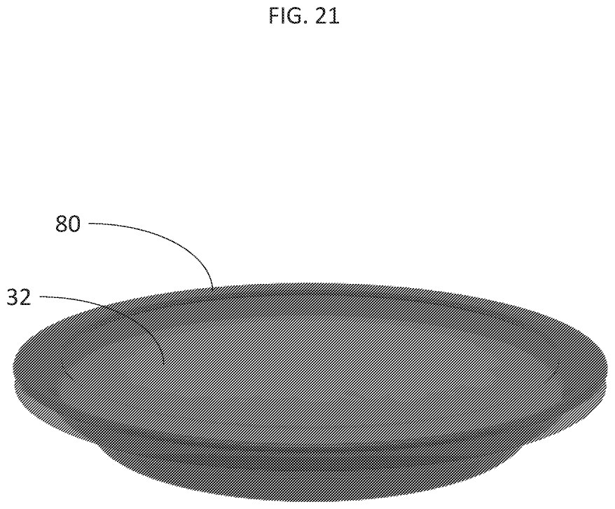

As depicted in FIGS. 19-21, the delivery device can further optionally include a lid 80 engageable with the housing 28. The lid 80 can be provided in addition to the barrier element 32, or can replace the barrier element 32. The lid can be selectively positioned coupled to the housing 28, and can facilitate a replaceable barrier to prevent volatile medium from emanating to an external environment while creating a sealed structure. The lid can include any suitable material, such as but not limited to plastic. With embodiments having a flange at the top rim of the housing, the lid can cooperate with the flange to provide a sealed configuration. The lid can be opaque, as shown in FIGS. 19-20, or can have a degree of transparency as shown in FIG. 21.

In accordance with another aspect of the disclosed subject matter, a method of using a volatile medium delivery system is provided. The method includes providing a volatile medium delivery device including a housing and a substrate having a volatile medium absorbed therein. The method further includes engaging the substrate with the housing and positioning the substrate with respect to the housing to control a predetermined release rate of volatile medium to the external environment.

In accordance with the embodiments of the subject matter previously described, the components of the volatile medium delivery device can be made out of a plurality of suitable materials. For instance, the substrate can be made of any suitable absorbent material, including paper, wood, polyethylene (e.g., porous polyethylene), nonwoven materials, fabrics (e.g., felted fabrics), foams (e.g., rigid foams), absorbent ceramics, paper substitutes such as Teslin® (PPG Industries), and combinations thereof. In certain embodiments, the substrate is made of paper. In other certain embodiments, the substrate is made of absorbent ceramic, such as depicted in the embodiment of FIGS. 15-16.

In particular embodiments, the substrate can be made from an absorbent rigid or semi-rigid paper such as that offered by the Robert Wilson Paper Corp. of Lynbrook, N.Y. By way of example, and not limitation, the thickness of the paper can range from 0.025 in to 0.060 in. The compatibility and integrity of paper and its ability to resist negative interaction with the volatile medium makes paper well-suited as a substrate. However, in certain embodiments, the substrate can include an absorbent ceramic material, for example, to impart strength and rigidity to the substrate and can include any suitable thickness dimension.

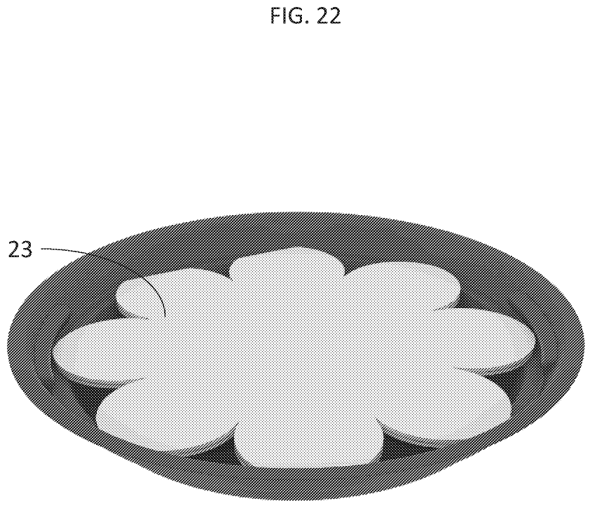

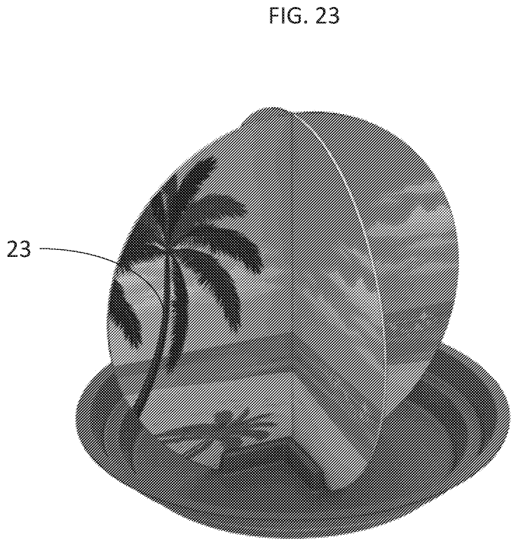

As embodied herein, the substrate and the corresponding housing can have any suitable shape and size. Although the substrates depicted in the accompanying figures are primarily geometric shapes, such as circles and rectangles, the substrate can have a non-geometric or irregular shape. For example, the substrate can be die-cut to communicate a holiday or other decorative theme. Further, the substrate can also include other desired shapes such as a flower shape, as depicted in FIG. 22. As such, the housing and the substrate can include different shapes and need not correspond to each other. Additionally or alternatively, the substrate can include any desired graphic, texture, copy, or colored design. For example, the substrate can include a tropical beach scene, as depicted in FIG. 23. Furthermore, the fragrance associated with a specific shape or depiction of the substrate can be associated with said configuration. For example and with respect to FIG. 23, a fragrance associated the substrate 23 having a tropical beach scene can be associated with a beach, such as Island Breeze or Beach Breeze fragrance as manufactured by Takasago International Corporation and Takasago International Corp. (USA).

As embodied herein, the volatile medium can include a fragrance, insecticide, medicant, decongestant, insect repellant, or a combination thereof. The volatile medium delivery system can release the volatile medium at the predetermined release rate for a certain period of time. For example, the volatile medium can be emanated from the substrate to the external environment for at least 1 hour, at least 24 hours, at least 7 days, at least 14 days, at least 21 days, or at least 28 days.

The volatile medium delivery system also provides an efficient method of dosing the substrate with the volatile medium. The amount of volatile medium loaded onto the substrate can vary depending on the intended application of the volatile medium delivery system. Further, when the substrate is packaged with the housing, the opening of the housing is substantially covered by the positioning of the substrate. Accordingly, certain known techniques, such as a high speed filling line, may be overly complicated to dose the substrate while it is contained in the housing. For example, the time required for the volatile medium to be absorbed fully by the substrate may be greater than the time between placing the substrate within the housing and applying the barrier element to the housing. Thus, the use of a high speed filling line to dose the system after inclusion of the substrate could unnecessarily delay the packaging process, particularly for larger fill volumes.

Thus, the presently disclosed subject matter provides a filling method where a suitable predetermined amount of the volatile medium is dosed into the housing prior to the placement of the substrate within the housing. The system is then sealed with the barrier element, e.g., via a heat sealable barrier laminate. The substrate within the housing can become dosed through the motion encountered during transit. To promote dosing, the system can be packaged in a master shipper in an upside down orientation so that the volatile medium within the housing comes into intimate contact with the substrate, if needed. Over time, the volatile medium will reach an equilibrium condition where the substrate is fully and uniformly saturated.

This method can provide an additional benefit as the dosing of the volatile medium into the housing prior to the insertion of the substrate can prevent the flange of the housing from being spoiled by the volatile medium and ensure that the heat sealing process proceeds without interference from the volatile medium.

While the disclosed subject matter is described herein in terms of certain preferred embodiments, those skilled in the art will recognize that various modifications and improvements can be made to the disclosed subject matter without departing from the scope thereof. Additional features known in the art likewise can be incorporated. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Furthermore, although reference is made to particular configurations of substrates, e.g., substrates having two members, throughout this disclosure, other suitable substrates can engage with the housing and support device of the system disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the system and method of the disclosed subject matter without departing from the spirit or scope of the disclosed subject matter. Thus, it is intended that the disclosed subject matter include modifications and variations that are within the scope of the appended claims and their equivalents.

What is claimed is:

1. A volatile medium delivery system, comprising:
a housing;
a barrier element engageable with the housing; and
a substrate having a volatile medium absorbed therein, wherein the substrate is engageable with the housing and positionable with respect to the housing to control a predetermined release rate of volatile medium to an external environment.

2. The volatile medium delivery system of claim 1, wherein the housing further includes a bottom surface, a sidewall coupled to the bottom surface, and a support device, wherein the substrate is engageable with the support device.

3. The volatile medium delivery system according to claim 2, wherein the sidewall further comprises a flange, wherein the barrier element is engageable with the housing about the flange.

4. The volatile medium delivery system according to claim 3, wherein the barrier element is removably coupled to the flange by heat seal.

5. The volatile medium delivery system according to claim 2, wherein at least a portion of the substrate is engageable with the sidewall.

6. The volatile medium delivery system according to claim 2, wherein the sidewall further comprises a projection and the substrate is engageable with the projection.

7. The volatile medium delivery system according to claim 2, wherein the support device includes at least a first structure and a second structure, wherein the first structure is disposed a predetermined distance dimension from the second structure to define a first channel therebetween, wherein the substrate is receivable in the channel.

8. The volatile medium delivery system according to claim 7, wherein a thickness dimension of the substrate is not greater than the predetermined distance dimension of the first channel.

9. The volatile medium delivery system according to claim 7, wherein the first structure includes a first wall and a second wall disposed the predetermined distance dimension from the first wall, wherein the second structure includes a first wall and a second wall disposed from the first wall of the second structure at the predetermined distance dimension, wherein each of the first wall of the first structure, the second wall of the first structure, the first wall of the second structure, and the second wall of the second structure comprise an L-shaped configuration to define a channel in the shape of a plus (+) symbol.

10. The volatile medium delivery system according to claim 2, wherein the substrate comprises a first member and a second member, wherein each of the first member and second member have a front surface and a back surface.

11. The volatile medium delivery system according to claim 10, wherein the first member is coupled to the second member by a hinge.

12. The volatile medium delivery system according to claim 10, wherein the first member defines a slit, wherein the first member is engageable with the second member by insertion of the second member into the slit of the first member.

13. The volatile medium delivery system according to claim 10, wherein the substrate is positioned in the support structure having the front surface of the first member and the front surface of the second member exposed to the external environment to control the predetermined release rate of volatile medium.

14. The volatile medium delivery system according to claim 10, wherein the substrate is positioned in the support device having the front and back surfaces of the first member and the front and back surfaces of the second member exposed to the external environment to effect a maximum release rate of volatile medium.

15. The volatile medium delivery system according to claim 2, wherein the housing further includes a handle device, wherein the handle device defines an aperture.

16. The volatile medium delivery system according to claim 1, further comprising a frame member, wherein the substrate is receivable in the frame member.

17. The volatile medium delivery system according to claim 16, wherein the frame member includes a hinge.

18. The volatile medium delivery system according to claim 1, wherein the volatile medium comprises at least one of a fragrance, insecticide, medicant, decongestant, insect repellant, or a combination thereof.

19. The volatile medium delivery system according to claim 1, wherein the substrate comprises at least one of paper, wood, polyethylene, fabric, foam, paper substitute, absorbent ceramic, or a nonwoven material.

20. The volatile medium delivery device according to claim 1, wherein the volatile medium is emanated from the substrate to the external environment at the predetermined release rate for at least one of 1 hour, 24 hours, 7 days, 14 days, 21 days, or 28 days.

21. A method of using a volatile medium delivery system, comprising:
providing a volatile medium delivery device including
a housing, and
a substrate having a volatile medium absorbed therein;
engaging the substrate with the housing; and
positioning the substrate with respect to the housing to control a predetermined release rate of volatile medium to the external environment.

22. The method of claim 21, wherein the substrate comprises a first member and a second member, wherein each of the first member and second member have a front surface and a back surface, and wherein the positioning includes engaging at least a portion of the substrate with the housing to expose at least one of the first member and the second member to the external environment.
